(19) United States
(12) Patent Application Publication
(10) Pub. No.: US 2013/0137776 A1
(43) Pub. Date: May 30, 2013

(54) METHOD FOR INHIBITING HISTONE GENE TRANSCRIPTION AND EXPRESSION

(71) Applicant: China Medical University, Taichung City (TW)

(72) Inventors: Tzong-Ming SHIEH, New Taipei City (TW); Cheng-Chia YU, Taoyuan City (TW); Shih-Min HSIA, New Taipei City (TW)

(73) Assignee: CHINA MEDICAL UNIVERSITY, Taichung City (TW)

(21) Appl. No.: 13/738,036

(22) Filed: Jan. 10, 2013

Related U.S. Application Data
(62) Division of application No. 13/299,837, filed on Nov. 18, 2011.

(30) Foreign Application Priority Data
Aug. 22, 2011 (TW) 100129942

(51) Int. Cl. A61K 31/122

(52) U.S. Cl. CPC A61K 31/122
USPC 514/690

(57) ABSTRACT

A method for inhibiting at least one of histone gene transcription and histone expression in a subject is provided. The method comprises administrating to the subject an effective amount of a compound of formula (I) or a pharmaceutically acceptable salt thereof:

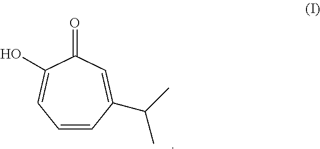

### CROSS-REFERENCES TO RELATED APPLICATIONS

This application is a divisional application of U.S. application Ser. No. 13/299,837 filed Nov. 18, 2011 which claims the benefit of Taiwan Patent Application No. 100129942, filed on Aug. 22, 2011, in the Taiwan Intellectual Property Office, the disclosures of which are incorporated herein in their entirety by reference.

### BACKGROUND OF THE INVENTION

1. Field of the Invention

The present invention relates to the inhibition of histone gene transcription and/or histone expression, especially in the inhibition of abnormal cell proliferation and curing keloids, oral submucous fibrosis (OSF), prostatic hyperplasia, and fibrocyst.

2. Descriptions of the Related Art

The cell cycle of a eukaryotic cell is the period of time from the end of a previous mitosis phase to the next one. In general, according to the physiological activity occurring in a cell, the cell cycle can be divided into the interphase and mitosis phase, wherein interphase occupies around 90% of the whole period of the cell cycle. In interphase, a cell replicates its DNA and synthesizes related proteins to finish the preparation before mitosis. In the mitosis phase, a cell evenly distributes cellular substances and divides to form two daughter cells. The interphase can be further divided into a period prior to the synthesis of DNA (gap 1 phase, G1 phase), DNA synthesis phase (S phase), and a period after the synthesis of DNA (gap 2, G2 phase). The mitosis phase can be further divided into prophase, prometaphase, metaphase, anaphase, and telophase.

Under normal conditions, most cells in an organism are in the resting state (i.e., G0 phase). When the cell is damaged or needs to be renewed, the cell starts the cell cycle process to carry out mitosis. The process of the cell cycle is regulated by various cyclins, and is strictly controlled by various regulation factors. Furthermore, there are several check points existing in the cell cycle. Once the function of the cyclins or the process of DNA replication is abnormal, the check points will be activated to arrest the cell cycle, which may even result in cell apoptosis to prevent the formation of abnormal cells or remove abnormal cells.

It has been found in the previous study that if the aforesaid regulation/control mechanism is ineffective in uncontrolled cell cycles, many diseases (such as cancer) will occur. This phenomenon occurs because cells cannot receive the signal to stop growth, causing unlimited proliferation and continuous dividing and stacking, resulting in the abnormality of the physiological activity in the body, and even affecting the functions of tissues and organs. Therefore, if the cell cycle of a cell of which the cell cycle is abnormal can be controlled or even blocked, the proliferation of the cell can be stopped, thereby achieving the purpose of curing related diseases. For example, it is known that alkylating agents can cause the alkylation of guanine to affect the base pairing between double-stranded DNA, thereby arresting the cell cycle in G0/G1 phase. In another aspect, nucleic acid analogues (such as methotrexate) can disrupt DNA replication by inserting itself in the structure of DNA, thereby arresting the cell cycle in the synthesis phase. All the aforesaid reagents are now used in clinical medical treatments.

However, most conventional approaches for inhibiting cell proliferation lack specificity to abnormally proliferating cells, and these approaches have higher adverse side effects because these approaches simultaneously kill normal as well. Therefore, there is still a need for an approach with specificity towards inhibiting the abnormal proliferation of cells to effectively cure diseases related to cell abnormal proliferation, and lower the side effects of the drug.

The present invention is a research for the above demand. The inventors of the present invention found that hinokitiol can specifically inhibit the gene transcription of histone involved in the cell cycle of abnormally proliferating cells and inhibit histone expression, thereby arresting the cell cycle in the synthesis phase. Therefore, hinokitiol can be used for curing the diseases related to cell abnormal proliferation.

### SUMMARY OF THE INVENTION

The primary objective of this invention is to provide a method for inhibiting at least one of histone gene transcription and histone expression in a subject, comprising administrating to the subject an effective amount of a compound of formula (I) or a pharmaceutically acceptable salt of the compound of formula (I):

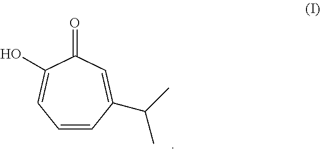

Another objective of this invention is to provide a pharmaceutical composition for inhibiting at least one of histone gene transcription and histone expression, which comprises an effective amount of the compound of formula (I) or a pharmaceutically acceptable salt of the compound of formula (I).

Yet a further objective of this invention is to provide use of the compound of formula (I) or a pharmaceutically acceptable salt of the compound of formula (I) in the manufacture of a medicament for inhibiting at least one of histone gene transcription and histone expression.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

The file of this patent contains at least one drawing executed in color. Copies of this patent with color drawings will be provided by the Patent and Trademark Office upon request and payment of the necessary fee.

FIG. 1 is a microarray analysis image showing the inhibition of hinokitiol on the histone gene transcription of the HSC-3 cancer cell line;

FIG. 2 is an electrophoresis picture showing the inhibition of hinokitiol on the histone gene transcription of the HSC-3 cancer cell line;

FIG. 3 is an electrophoresis picture showing the inhibition of hinokitiol with different concentrations on the histone expression of the HSC-3 cancer cell line and SAS cancer cell line;

FIG. 4A is a flow cytometry picture showing the influence of hinokitiol with different concentrations on the cell cycle of the HSC-3 cancer cell line; and

FIG. 4B is a statistical column diagram showing the influence of hinokitiol with different concentrations on the cell cycle of the HSC-3 cancer cell line.

### DETAILED DESCRIPTION OF THE INVENTION

Unless otherwise stated herein, the terms “a (an)”, “the” or the like used in this specification (especially in the claims hereinafter) shall be understood to encompass both the singular and plural forms.

DNA synthesis phase (S phase) is a critical stage in the cell cycle. A proliferating cell will finish its DNA replication in this stage. When the DNA replication is finished, the DNA content in the cell will increase by two folds. At the same time, the cell will synthesize/express essential proteins required for the process of the cell cycle in the synthesis phase, especially express histone protein, which can help the assembly of replicated DNA to be folded into a chromosomal structure.

Histone is an alkaline protein which can bind with the DNA of a eukaryotic cell, and is a basic structural protein in the chromosome of a cell. According to the sequence, structure, and function, histone can be generally classified into five types: H1, H2A, H2B, H3, and H4. All histones contain abundant alkaline amino acids with positive charges, and thus, they can bind with DNA with negative charges to form a stable chromosomal structure. Histone H2A, H2B, H3, and H4 can assemble into an octameric structure, and they can be winded by DNA in a supercoiled form to form a nucleosome core particle. Histone H1 is a connecting histone, which can connect the nucleosome core particles with the entry site and exit site of the DNA molecule, thereby making the chromosomal structure more stable, or form a more compact and higher-level structure.

A common characteristic of abnormally proliferating cells is that the ratio of cells in DNA synthesis phase increases. Since histone is a critical protein in the synthesis phase, the inhibition of histone gene transcription and/or histone expression disrupts the formation of the chromosomal structure, thus arresting the cell cycle in the synthesis phase and inhibiting abnormal cell proliferation can be achieved.

The inventors of the present invention found that hinokitiol with the following formula (I) has the activity to specifically inhibit histone gene transcription and histone expression in abnormally proliferating cells:

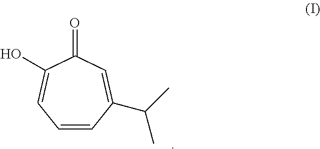

Therefore, the present invention provides a method for inhibiting at least one of histone gene transcription and histone expression in a subject, comprising administrating to the subject an effective amount of a compound of formula (I) or a pharmaceutically acceptable salt of the compound of formula (I) (i.e., the active component of the present invention):

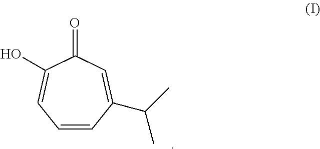

Examples of the pharmaceutically acceptable salt of the compound of formula (I) include, but are not limited to, alkali metal salts, such as sodium salts and potassium salts; alkali earth metal salts, such as calcium salts, magnesium salts, and barium salts; transition metal salts, such as zinc salts, copper salts, iron salts, cobalt salts, titanium salts, and vanadium salts; aluminum salts; tin salts; alkanolamine salts, such as diethanolamine salts, 2-amino-2-ethyl-1,3-propanediol salts and triethanolamine salts; heterocyclic amine salts, such as morpholine salts, piperazine salts, and piperidine salts; and basic amine salts, such as ammonium salts, arginine salts, lysine salts, and histidine salts. In the method of the present invention, the active component is preferably the compound of formula (I). Preferably, in the method of the present invention, the subject is a mammalian, and more preferably, the subject is a human.

The compound of formula (I) (i.e., hinokitiol) is an active component obtained from the extracts of plants of Cypress Family. It is known that the compound of formula (I) and its salts have the effects of sterilization, anti-bacteria, and antisepsis, and have low biological toxicity (which can be seen in Imai, N. et al., Lack of hinokitiol (beta-thujaplicin) carcinogenicity in F344/DuCrj rats. J Toxicol Sci, 2006. 31(4): p. 357-70; and Ema, M. et al., Evaluation of developmental toxicity of beta-thujaplicin (hinokitiol) following oral administration during organogenesis in rats. Food Chem Toxicol, 2004. 42(3): p. 465-70, which are entirely incorporated hereinto by reference). As shown in the following examples, the method of the present invention can effectively inhibit histone gene transcription and histone expression in abnormally proliferative cells, and especially can effectively inhibit gene transcription and/or expression of at least one of histone selected from a group consisting of histone H1, histone H2A, histone H2B, and histone H3.

The method of the present invention can inhibit the formation of histone by inhibiting histone gene transcription and/or histone expression, thereby inhibiting the formation of the chromosomal structure and arresting the cell cycle of abnormally proliferative cells at the synthesis phase to achieve the effect of inhibiting abnormal cell proliferation.

Currently, the common diseases related to abnormal cell proliferation include keloid, oral submucous fibrosis (OSF), prostatic hyperplasia, fibrocyst, etc. The cause of Keloid usually relates to individual physiques or inherent factors. If a patient with keloid has a wound in which the dermis is damaged, abnormal cell proliferation will occur in the regenerative tissues of the wound, and thus leads to hypertrophic scars. Oral submucous fibrosis is a disease caused by the abnormal proliferation of fibroblasts in oral connective tissues and the over-accumulation of collagen. The disease may be caused by stimulation to oral mucosa induced by the components in betel nuts (such as alkaloids) or drugs. This stimulation will induce the abnormal proliferation of fibroblasts and the over-accumulation of collagen, causing the fibrosis of the oral mucosa of the patient to affect the normal function of oral cavity. Prostatic hyperplasia is usually caused by the imbalance of androgen hormones, leading to the abnormal proliferation of prostatic cells. Fibrocyst is usually induced by the imbalance of female hormones (such as estrogen or progesterone), causing the proliferation of cystic cells. Fibrocysts usually occur in females aged thirty to fifty, especially in menopausal women.

The method of the present invention can effectively inhibit abnormal cell proliferation, and thus, it can be used for curing diseases related to abnormal cell proliferation, such as keloids, oral submucous fibrosis, prostatic hyperplasia, fibrocysts, etc. Since hinokitiol has effects of anti-bacteria, anti-oxidation, and anti-inflammation (which can be seen in MORITA YASUHIRO et al., Light stability and Antibacterial activities of metal chelates of Hinokitiol. Symposium Papers. Symposium on the Chemistry of Natural Products, 1998, VOL. 40^{th}, P. 529-533; U.S. Pat. No. 7,294,609 B2; and U.S. Pat. No. 6,387,417 B1, which are entirely incorporated hereinto by reference), the active component of the present invention also can be added into mouthwash formulas, a periodontal regeneration membrane, anti-adhesion membranes for surgery, dental cavity filling materials, or medical equipments to achieve multiple effects of inhibiting abnormal cell proliferation, anti-bacteria, anti-oxidation, anti-inflammation, etc.

In the method of the present invention, the aforesaid active component can be administrated as a medicament. Therefore, the present invention also provides a pharmaceutical composition for inhibiting histone gene transcription and/or expression. The pharmaceutical composition comprises a pharmaceutically acceptable carrier and an effective amount of the active component.

The pharmaceutical composition of the present invention can be used in both veterinary and human medicine, and it can be in any suitable form and can be administrated by any suitable manner without particular limits. For example, but not limited thereby, the pharmaceutical composition can be applied by oral administration, subcutaneous injection, intravenous injection, etc. Depending on the form and purpose of the pharmaceutical composition of the present invention, the pharmaceutical composition may comprise a pharmaceutically acceptable carrier.

For manufacturing a medicament suitable for oral administration, the pharmaceutical composition of the present invention can comprise a pharmaceutically acceptable carrier which has no adverse influence on the activity of hinokitiol, such as solvents, oily solvents, thinners, stabilizers, absorption delaying agents, disintegrants, emulsifiers, antioxidants, binders, lubricants, moisture absorbents, etc. The pharmaceutical composition can be prepared in a form suitable for oral administration, such as a tablet, a capsule, a granule, powder, a fluid extract, a solution, syrup, a suspension, an emulsion, a tincture, etc., by any suitable approach.

As for a medicament suitable for subcutaneous or intravenous injection, the pharmaceutical composition of the present invention can comprise one or more components, such as an isotonic solution, a saline buffer solution (such as a phosphate buffer solution or a citrate buffer solution), a solubilizer, an emulsifier, other carriers, etc., to produce an intravenous injection, an emulsion intravenous injection, a powder injection, a suspension injection, a powder-suspension injection, etc.

Optionally, in addition to the above adjuvants, other additives, such as a flavoring agent, a toner, a coloring agent, etc., can be added to the pharmaceutical composition of the present invention to enhance the taste and visual appeal of the composition. A suitable amount of a preservative, a conservative, an antiseptic, an anti-fungus reagent, etc., also can be added to improve the storability of the resultant medicament.

The pharmaceutical composition may optionally combine one or more other active components to enhance the effect of the medicament or increase the flexibility for the formulation, as long as the other active components have no adverse effect on hinokitiol. Moreover, depending on the requirements of the subject, the pharmaceutical composition of the present invention can be applied with various administration frequencies, such as once a day, several times a day, or once for days, etc. For example, in the method of the present invention, when applied to the human body for inhibiting abnormal cell proliferation, the dose of the pharmaceutical composition or the active component (i.e., the compound of formula (I) or a pharmaceutically acceptable salt thereof) of the present invention (as the compound of formula (I)) is about 15 mg/kg-body weight to about 20 mg/kg-body weight per day, wherein the unit “mg/kg-body weight” means the dosage required per kg-body weight. However, for patients with acute conditions (e.g., severe abnormal cell proliferation), the dosage can be increased to several times or several tens of times, depending on practical requirements.

The present invention also provides use of the compound of formula (I) and/or its pharmaceutically acceptable salt in the manufacture of a medicament for inhibiting at least one of histone gene transcription and histone expression. Specifically, the medicament can be used for inhibiting histone gene transcription and/or histone expression of at least one of the histones selected from a group consisting of histone H1, histone H2A, histone H2B, and histone H3. Based on the inhibitory activity of the compound of formula (I) on histone gene transcription and histone expression, the medicament especially can be used for inhibiting abnormal cell proliferation by arresting the cell cycle in the synthesis phase, thereby curing diseases, such as keloids, oral submucous fibrosis, prostatic hyperplasia, fibrocysts, etc.

The detailed technology and preferred embodiments implemented for the present invention are described in the following paragraphs; however, the scope of the present invention is not limited thereby.

### EXAMPLE

### Example 1

### Microarray Analysis

(1) Cell Culture and RNA Collection

Human oral squamous-cell carcinoma (OSCC) cell lines, such as HSC-3 (JCRB0623, Japan), was cultured. The relative culture methods can be seen in Shieh, T. M. et al., Association of expression aberrances and genetic polymorphisms of lysyl oxidase with areca-associated oral tumorigenesis. Clin Cancer Res, 2007. 13(15 Pt 1): p. 4378-85; Chen, J. C. et al., Gypenosides induced G0/G1 arrest via CHk2 and apoptosis through endoplasmic reticulum stress and mitochondria-dependent pathways in human tongue cancer SCC-4 cells. Oral Oncol, 2009. 45(3): p. 273-83; and Lin, C. C. et al., Berberine induces apoptosis in human HSC-3 oral cancer cells via simultaneous activation of the death receptor-mediated and mitochondrial pathway. Anticancer Res, 2007. 27(5A): p. 3371-8, which are entirely incorporated hereinto by reference.

Then, the HSC-3 cell lines were divided into four groups: two groups of the cells were not treated by hinokitiol (0 μM); one group of the cells was treated by 6.25 μM hinokitiol (commercially purchased from Sigma-Aldrich company); and one group of the cells was treated by 12.5 μM hinokitiol. After being cultured for 24 hours, the cells were scratched from the cell culture dish and collected by centrifugation. The total RNAs were isolated by the Tri-reagent (commercially purchased from Applied Biosystem). The content and purity of the obtained RNA were respectively determined at wavelengths of 260 nm and 280 nm by using a micro-volume spectrophotometer (Nanodrop ND-1000, commercially purchased from Labtech. International company). Each sample (300 ng) was amplified and labeled by using GeneChip WT Sense Target Labeling and Control Reagents (commercially purchased from Affymetrix company) to analyze the expression level.

(2) Hybridization Test

The above obtained RNA samples were put in an Affymetrix GeneChip Human Gene 1.0 ST microarray to perform a hybridization test. The group of the HSC-3 cells that were not treated by hinokitiol was used as a control group. The testing was performed by hybridization at 45° C. under the shaking speed of 60 rpm for 17 hours. Then, the microarray was washed by an Affymetrix Fluidics Station 450 system, stained by streptavidin-phycoerythrin (GeneChip® Hybridization, Wash, and Stain Kit, 900720), and scanned by Affymetrix GeneChipR Scanner 3000. The obtained data was analyzed by Expression Console software (commercially purchased from Affymetrix) with default Robust multi-array average (RMA) parameters. The genes of which expression was changed by above two-fold in the cells after the treatment of hinokitiol were selected. The results are shown in FIG. 1 and Table 1.

In FIG. 1, the red sections in the microarray indicate that the expression level of the genes was increased, and the green sections indicate that the expression level of the genes was decreased (i.e., compared with the control group in which the HSC-3 cells were not treated by hinokitiol). The corresponding gene name for each probe set ID on the microarray is shown in Table 1. As shown in FIG. 1 and Table 1, after the HSC-3 cancer cell lines were treated by hinokitiol, almost all the genes of which expression was decreased by above two-fold in the cells relate to histone, such as the gene H1d, H3g, H3j, H3a, H3h, H2bm, H2bg, H3l, H2bf, and H2a. This result indicates that hinokitiol can inhibit histone gene transcription of cancer cells.

TABLE 1

| Probe set ID | Gene abbreviation (full name) |
|---|---|
| 7931810 | KLF6 (Kruppel-like factor 6) |
| 8069676 | ADAMTS1 |
| | (ADAM metalloproteinase with thrombospondin motifs) |
| 8135069 | SERPINE1 (serpin peptidase inhibitor, clade E) |
| 7974425 | SAMD4A (sterile alpha motif domain containing 4A) |
| 8020551 | LAMAS (laminin, alpha 3) |
| 7949540 | C11orf68 (chromosome 11 open reading frame 68) |
| 8116372 | RNF130 (ring finger protein 130) |
| 8054054 | ANKRD36B (ankyrin repeat domain 36B) |
| 8090852 | AMOTL2 (angiomotin-like protein 2) |
| 8124430 | HIST1H1D (histone cluster 1, H1d) |
| 8124440 | HIST1H3G (histone cluster 1, H3g) |
| 8124537 | HIST1H3J (histone cluster 1, H1j) |
| 8117330 | HIST1H3A (histone cluster 1, H3a) |
| 8117589 | HIST1H3H (histone cluster 1, H3h) |
| 8117594 | HIST1H2BM (histone cluster 1, H2bm) |
| 8124423 | HIST1H2BG (histone cluster 1, H2bg) |
| 8124531 | HIST1H3I (histone cluster 1, H3i) |
| 8117395 | HIST1H2BF (histone cluster 1, H2bf) |
| 7924888 | HIST3H2A (histone cluster 3, H2a) |
| 7994109 | PLK1 (Polo-like kinase 1 (Drosophila)) |

### Example 2

### Reverse Transcription-PCR

HSC-3 cell lines were cultured and respectively treated by 0 μM and 6.25 μM hinokitiol for 24 hours. Then, the cells were scratched from the cell culture dishes and collected by centrifugation. Afterwards, reverse transcription-PCR (RT-PCR) was performed. The protocols and conditions related to RT-PCR can be seen in Shieh, T. M. et al., Association of expression aberrances and genetic polymorphisms of lysyl oxidase with areca-associated oral tumorigenesis. Clin Cancer Res, 2007. 13 (15 Pt1), p. 4378-85; and Shieh, T. M. et al., Association between the polymorphisms in exon 12 of hypoxia-inducible factor-1alpha and the clinicopathological features of oral squamous cell carcinoma. Oral Oncol, 2010. 46 (9), p. e47-53, which are entirely incorporated hereinto by reference.

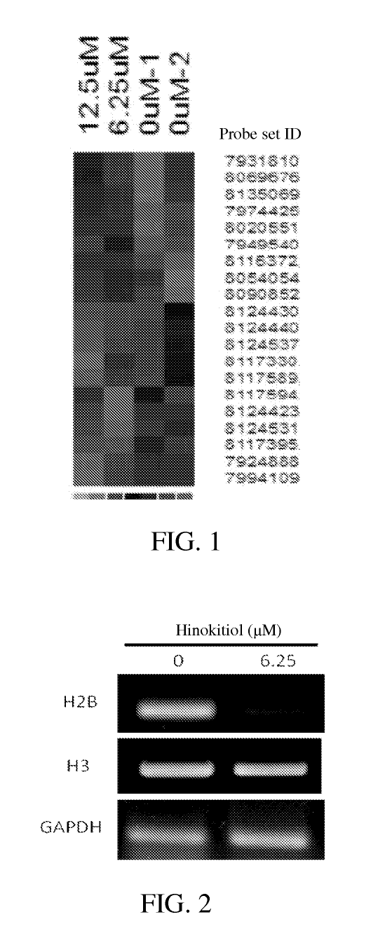

The gene expression level of Glyceraldehyde-3-phosphate dehydrogenase (GAPDH) was used as an internal control. The variation of the gene expression level of histone H2B and H3 in HSC3 cell lines treated by hinokitiol was compared with the control group, and the results are shown in FIG. 2. At least two independent RT-PCR experiments were performed to validate the reproducibility of the analysis.

The result shown in FIG. 2 indicates that after the HSC-3 cell line was treated by hinokitiol, the gene expression level of histone H2B and H3 was decreased. This result confirms the result of the microarray analysis in Example 1, i.e., hinokitiol can inhibit histone gene transcription of cancer cells.

### Example 3

### Western Blotting Analysis

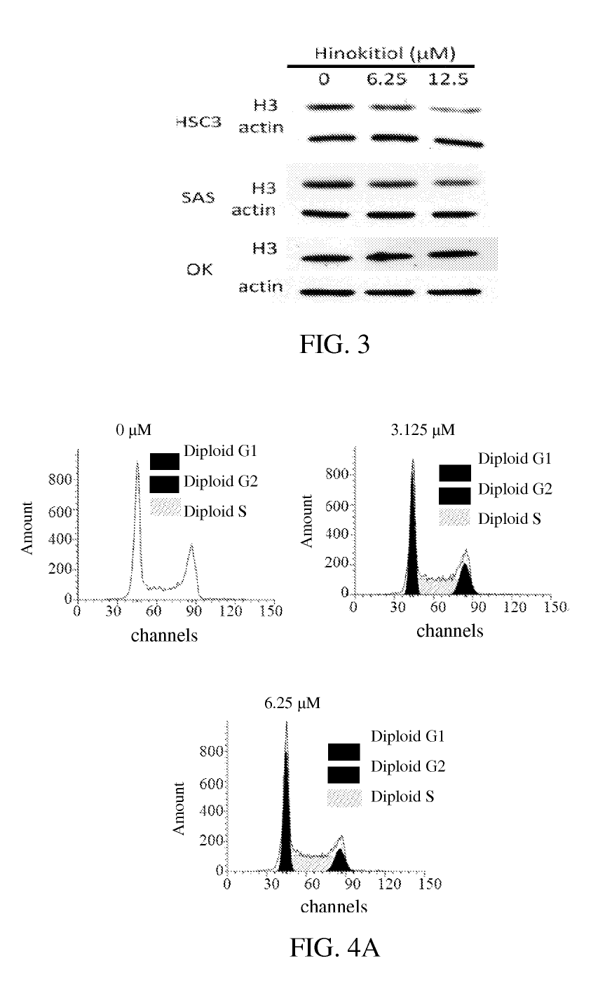

Normal human oral keratinocyte cell lines (OK, obtained from China Medical University Hospital, Taiwan), HSC-3 cancer cell lines, and SAS cancer cell lines (OSCC cancer cell line, obtained from the tongue tissue of a patient in National Yang Ming University, Taiwan) were cultured (the protocols related to cell culture can be seen in Lu, S. Y. et al., Ripe areca nut extract induces G1 phase arrests and senescence-associated phenotypes in normal human oral keratinocyte. Carcinogenesis, 2006. 27 (6), p. 1273-84; and Shieh, T. M. et al., Association of expression aberrances and genetic polymorphisms of lysyl oxidase with areca-associated oral tumorigenesis. Clin Cancer Res, 2007. 13 (15 Pt 1), p. 4378-85, which are entirely incorporated hereinto by reference). The cells were respectively treated by 0 μM, 6.25 μM, and 12.5 μM hinokitiol for 24 hours, and scratched from the cell culture dishes and collected by centrifugation. Then, western blotting analysis was performed (see Lu, S. Y. et al., Ripe areca nut extract induces G1 phase arrests and senescence-associated phenotypes in normal human oral keratinocyte. Carcinogenesis, 2006. 27 (6), p. 1273-84, which is entirely incorporated hereinto by reference). In this western blotting analysis, an H3 primary antibody (#9715, commercially purchased from Cell Signaling Technology®, U.S.A.) was diluted (1:2000) with a phosphate-buffered saline (PBS); and an actin primary antibody (MAB1501, commercially purchased from CHEMICON, U.S.A.) was diluted at 1:5000; a goat anti-rabbit secondary antibody (sc2004, commercially purchased from Santa Cruz Biotech company) was diluted at 1:5000; and a goat anti-mouse secondary antibody (sc2005, commercially purchased from Santa Cruz Biotech company) was diluted at 1:2000. The expression level of actin is used as an internal control for the analysis. The variation of the expression level of histone H3 in the cells treated by hinokitiol was compared with the control group, and the result is shown in FIG. 3.

As shown in FIG. 3, after being treated by hinokitiol, the expression level of histone H3 in the HSC-3 cancer cell line and the SAS cancer cell line was significantly decreased. However, the expression level of histone H3 in the normal human oral keratinocyte cell line was not affected. These results indicate that hinokitiol has a specific inhibitory effect on the expression level of histone H3 in abnormally proliferating cells.

### Example 4

### Cell Cycle Analysis

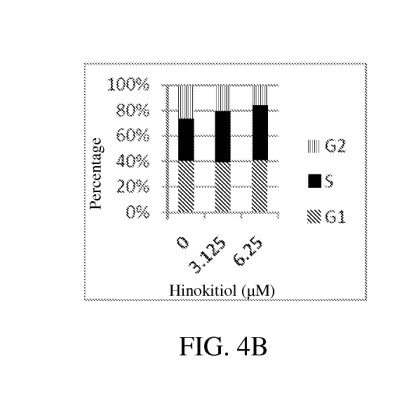

HSC-3 cells (2×10⁵/4 ml) were respectively treated by 0 μM, 3.12 μM, and 6.25 μM hinokitiol for 24 hours, washed by an ice-cold PBS buffer solution twice, and fixed in 70% ice-cold ethanol at 4° C. for 4 hours. Then, the cells were stained by propidium iodide (PI), and a cell cycle analysis was performed by using a BD FACSAria flow cytometer (commercially purchased from Becton Dickinson, Germany). The obtained data was analyzed by MULTICycle Software (commercially purchased from Coulter company, U.S.A.). The results are shown in FIGS. 4A and 4B.

FIG. 4A shows the analysis data of flow cytometry, indicating the amount of cells in the G1 phase, synthesis phase (S phase), and G2 phase after HSC-3 cells were treated by various concentrations of hinokitiol. FIG. 4B shows the statistical result of the data of flow cytometry, indicating the proportions of the cells in different phases of the cell cycle. These results show that after HSC-3 cells were respectively treated by 3.125 μM and 6.25 μM hinokitiol, the proportions of the cells in the synthesis phase were increased from 31.38% (cells treated by 0 μM hinokitiol) to 41.19% (cells treated by 3.125 μM hinokitiol) and 43.89% (cells treated by 6.25 μM hinokitiol). Moreover, these results indicate that hinokitiol indeed can arrest proliferating cells in the synthesis phase by inhibiting histone expression of the cells, thereby achieving the effect of inhibiting abnormal cell proliferation.

The results of Examples 1 to 4 indicate that the method of the present invention can specifically inhibit histone gene transcription and/or histone expression in abnormally proliferating cells, making histone unable to form, thereby, arresting the cell cycle of abnormally proliferating cell at the synthesis phase to achieve the effect of inhibiting abnormal cell proliferation. Therefore, the method of the present invention can be used for curing diseases related to abnormal cell proliferation, such as keloids, oral submucous fibrosis, prostatic hyperplasia, fibrocysts, etc.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A method for curing keloid by inhibiting at least one of histone gene transcription and histone expression in a subject, comprising administrating to the subject an effective amount of a compound of formula (I) or a pharmaceutically acceptable salt of the compound of formula (I):

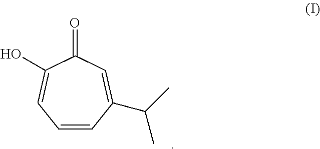

2. The method as claimed in claim 1, which comprises administrating to the subject an effective amount of the compound of formula (I).

3. The method as claimed in claim 1, which is for inhibiting histone gene transcription, wherein the histone is selected from a group consisting of histone H1, histone H2A, histone H2B, histone H3, and combinations thereof.

4. The method as claimed in claim 2, which is for inhibiting histone gene transcription, wherein the histone is selected from a group consisting of histone H1, histone H2A, histone H2B, histone H3, and combinations thereof.

5. The method as claimed in claim 1, which is for inhibiting histone expression, wherein the histone is selected from a group consisting of histone H1, histone H2A, histone H2B, histone H3, and combinations thereof.

6. The method as claimed in claim 2, which is for inhibiting histone expression, wherein the histone is selected from a group consisting of histone H1, histone H2A, histone H2B, histone H3, and combinations thereof.

7. The method as claimed in claim 1, which is for arresting cell cycle in synthesis phase (S phase).

8. The method as claimed in claim 2, which is for arresting cell cycle in synthesis phase.

9. The method as claimed in claim 7, which is for inhibiting abnormal cell proliferation.

10. The method as claimed in claim 8, which is for inhibiting abnormal cell proliferation.

11. The method as claimed in claim 9, wherein the dosage of the compound of formula (I) or a pharmaceutically acceptable salt of the compound of formula (I) (as the compound of formula (I)) is about 15 mg/kg-body weight to about 20 mg/kg-body weight.

12. The method as claimed in claim 10, wherein the dosage of the compound of formula (I) or a pharmaceutically acceptable salt of the compound of formula (I) (as the compound of formula (I)) is about 15 mg/kg-body weight to about 20 mg/kg-body weight.

13. The method as claimed in claim 1, wherein the subject is a mammalian.

14. The method as claimed in claim 13, wherein the mammalian is a human.
